(19) United States
(12) Patent Application Publication
(10) Pub. No.: US 2022/0125114 A1
(43) Pub. Date: Apr. 28, 2022

(54) ATOMIZATION CORE

(71) Applicant: Shanghai QV Technologies Co., Ltd., Shanghai (CN)

(72) Inventors: Xiaofeng Peng, Oakville (CA); Qiwen Peng, Oakville (CA)

(21) Appl. No.: 17/573,151

(22) Filed: Jan. 11, 2022

Related U.S. Application Data
(63) Continuation of application No. PCT/CN2020/108894, filed on Aug. 13, 2020.

(30) Foreign Application Priority Data
Aug. 13, 2019 (CN) 201921301938.1

(51) Int. Cl. A24F 40/46; A24F 40/20; A24F 40/70

(52) U.S. Cl. CPC A24F 40/46; A24F 40/70; A24F 40/20

(57) ABSTRACT

The disclosure discloses an atomization core comprising a substrate, wherein a film with low oxygen content is deposited on the substrate, a passive film is deposited on this film with low oxygen content, the substrate is formed with fluidic transferring channels, and electrodes are formed on both ends of the substrate. The material of the substrate of the atomization core is monocrystalline alumina. A film with low oxygen content and a passive film are deposited on the substrate. The diameter of perforations of the fluidic transferring channels in the substrate is less than 250 μm. The spacing between walls of adjacent perforations of the fluidic transferring channels is less than 500 μm. Both the diameter and number of the perforations are controllable.

### CROSS-REFERENCES

This application is a continuation application of International Application No. PCT/CN2020/108894, filed Aug. 13, 2020, and entitled “Novel Atomization Core,” which claims priority to Chinese Application CN 201921301938.1, filed Aug. 13, 2019, and entitled “Novel Atomization Core.” The contents of all prior applications are hereby incorporated by reference in their entirety.

### TECHNICAL FIELD

The disclosure relates to atomization applications. More specifically, the disclosure relates to an atomization core.

### BACKGROUND

Atomization of liquid by resistance heating to generate aerosol is a common atomization method for e-cigarettes and some medical atomizers.

At present, electrical resistance heating is normally employed in e-cigarettes and some medical atomizers to heat liquids to generate aerosol. There are four types in general.

First, glass fiber rope plus heating wire: the most common e-cigarette atomizer generally winds the resistance heating wire on the fiber rope for transferring liquid. The glass fiber rope was used as the main liquid transferring material because of its firm selvage, high temperature resistance, strong liquid absorption, and fast transferring speed. However, the biggest disadvantage of glass fiber rope is that it is easy to fall off and produce flocs. In addition, when the heating wire is wound around the fiber rope and the position of the heating wire is fixed, the surface of the heating wire is exposed to the outside of fiber rope, which results in low consistency of the atomizing device, low atomizing efficiency, and dry burning.

Second, cotton plus heating wire: around 2013, cotton began to replace glass fiber rope as the main e-juice transferring material. Compared with glass fiber rope, cotton is safer and delivers a more tabacco-authentical taste. Its development has gone from absorbent cotton and organic cotton to professional e-cigarette cotton such as highest grade long-staple cotton. At present, cotton plus heating wire is still the mainstream in the market, but sugar in the e-liquid will be adsorbed on the heating wire to generate low temperature coking, which leads to the darkening of cotton.

Third, ceramic atomization core: the development of e-cigarette has boomed to the emergence of various e-juice transferring materials. Porous ceramics have become popular for closed e-cigarettes. There are mainly two kinds of ceramic atomization cores on the market: one is to embed heating wires in a porous ceramic body, e.g., CCell™; the other is to screen print a layer of conductive heating wires on the porous ceramic, e.g., Feelm™ and Silmo™. The perforations of the porous ceramic are dispersed in various sizes, resulting in easily coking or dry burning of some liquid components during heating, or leakage of liquid due to large perforations. CN20188001973.3 has disclosed that a 0.5-5 μm thick titanium-zirconium alloy film and a 0.1-1 μm thick Au—Ag alloy protective film are sputtered and deposited on the porous ceramic. At this thickness, the film quality is inevitably affected by the surface roughness of the porous ceramic.

Fourth, other atomization cores: for example, CN201620757596.4, CN201810009220.9 and CN201910229470.8 have disclosed monocrystalline silicon-based MEMS atomization cores, which are expected to solve the problems of inconsistent atomizing temperature and flavor change caused by direct contact between the heating surface and the e-liquid. A micro-perforation plate with micro-perforation array is used to control the liquid flow. The diameters of the microfluidic channels are 10 to 500 μm, and those of the micro-perforation channels are 500 to 1000 μm. The metal films are one or more of Ti/Pt/Au, TiW/Au, Al, Cr or Pt/Au with a thickness of 200 to 500 nm. However, the system reliability of such devices is still at stake. Another example is CN201821218626.X and CN201810855337.9, which describe an atomizer of capillary array using stainless-steel medical tubes and glass tubes with inner diameters of 0.01-0.1 mm as capillaries. The external stainless-steel sheet is directly heated, thus similarly avoiding the contact between the heating body and e-liquid. The effective atomization area where the fluid passes through reaches up to 50%. These patents claim to have overcome the shortcomings of ceramic heating bodies, thus achieving atomized e-cigarette closer to traditional cigarettes. However, the processing and assembly of micro-tubes pose certain safety risks for powder and other particles to enter the aerosol.

### BRIEF SUMMARY

The disclosure aims to provide an atomization core to overcome the weaknesses in the above-mentioned disclosures.

For the above purpose, the disclosure provides an atomization core comprising a substrate, wherein a heating layer is deposited on the substrate, fluidic transferring channels are formed in the substrate, a diameter of the perforations of the fluidic transferring channels is less than 250 μm, and an array of the perforations of the fluidic transferring channels is arranged in a close-packed triangular or rectangular shape, a spacing between walls of adjacent perforations of the fluidic transferring channels is less than 500 μm, and electrodes are formed on both ends of the substrate.

Preferably, the heating layer is a pure metal film, an alloy film or a film with low oxygen content, the film with low oxygen content is a titanium oxide film or tantalum oxide film, and the thickness of the film with low oxygen content is 0.35 μm to 5 μm.

Preferably, a passive film is deposited on the heating layer.

Preferably, the passive film is made of an inert metal or alloy or compound film, preferably an Au film, and the thickness of the passive film is 10 nm to 50 nm.

Preferably, the diameter of the perforations of the fluidic transferring channels is less than 250 μm, the array of the perforations of the fluidic transferring channels is arranged in a close-packed triangular or rectangular shape, and the spacing between walls of adjacent perforations of the fluidic transferring channels is less than 500 μm.

Preferably, the electrodes are made of a safe conductive paste.

Preferably, a groove is formed in the back of the substrate, the groove being connected to the fluidic transferring channels.

Preferably, the substrate material is one of monocrystalline alumina, monocrystalline silicon or polycrystalline silicon, or a dense ceramic material such as alumina, zirconia or silica ceramics.

### BRIEF DESCRIPTION OF THE DRAWINGS

FIG. 1 is a schematic view of an atomization core of the disclosure;

FIG. 2 is a schematic top view of the atomization core of the disclosure as described in Embodiment 1;

FIG. 3 is a schematic sectional view of the atomization core of FIG. 2;

FIG. 4 is an enlarged schematic view of the atomization core as shown in Position A in FIG. 2;

FIG. 5 is a schematic top view of an atomization core of the disclosure as described in Embodiment 2;

FIG. 6 is a schematic sectional view of the atomization core of FIG. 5; and

FIG. 7 is an enlarged schematic view of the atomization core as shown in Position B in FIG. 5.

### DETAILED DESCRIPTION

The disclosure provides an atomization core having a substrate made of monocrystalline alumina, and a film with low oxygen content and a passive film deposited on the substrate. The diameter of the perforations of the fluidic transferring channels in the substrate is 120 μm, 100 μm, 80 μm or 60 μm. The array of the perforations of the fluidic transferring channels is arranged in a close-packed triangle, or in a close-packed rectangular shape or other shapes. The spacing between the walls of the adjacent perforations of the fluidic transferring channels is 250 μm, 200 μm, 150 μm or 100 μm and the diameter and number of the perforations are controllable.

The electrodes are connected to a battery. The film with low oxygen content and the passive film deposited between the walls of the adjacent perforations form a uniform temperature field and a uniform aerosol nucleation center, meanwhile the liquid flow and the air flow are also controlled during the atomization process, and consequently the generated aerosol is also controlled to achieve the better nicotine delivery efficiency and various atomization satisfactions. Furthermore, the film with low oxygen content and the passive film are deposited on the substrate, so that the non-porous areas are the heating surface. Combined with the controllable perforation diameters of the fluidic transferring channels, uniform atomization is built in for different e-liquids and other liquids, without coking, ceramic particle emission or any heavy metals.

In the following, embodiments of the present disclosure will be described clearly and sufficiently with reference to the accompanying drawings. Obviously, the embodiments described are only exemplary and other embodiments may fall within the protection scope of the present disclosure.

### Embodiment 1

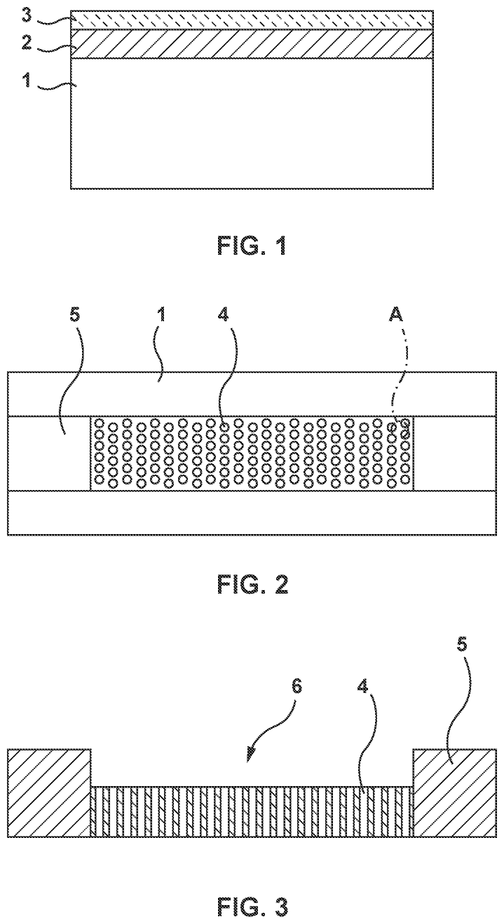

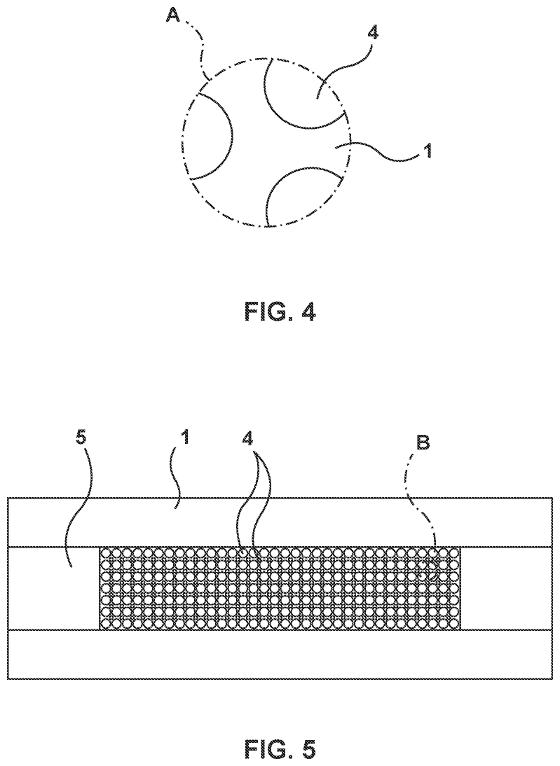

Referring to FIGS. 1-4, the disclosure provides an atomization core comprising a substrate (1) made of monocrystalline alumina, on which is deposited a heating layer (2), namely a film with low oxygen content, which is a titanium or tantalum oxide film. The thickness of the film with low oxygen content is 4.5 μm, on which is deposited a passive film (3), namely an inert metal or alloy film (an Au film in this embodiment). The thickness of the passive film (3) is 12 nm. The substrate (1) is formed with fluidic transferring channels (4) therein. The diameter of each of the perforations of the fluidic transferring channels (4) is 120 μm, which is processed by laser or drilled mechanically. The array of the perforations of the fluidic transferring channels (4) is arranged in a close-packed triangular shape. The spacing between the walls of the adjacent perforations of the fluidic transferring channels is 250 μm.

Electrodes (5) are formed on both ends of the substrate (1). The electrodes are made of a safe conductive paste and connected to a battery. The back of the substrate (1) is provided with a groove (6) which is connected to the fluidic transferring channel (4). The film with low oxygen content and the passive film (3) deposited between the walls of the adjacent perforations form a uniform temperature field and uniform vapor nucleation center. Combining controllable liquid fluidic and air fluidic channels in the atomization process, the aerosol generation is also controlled to achieve better nicotine delivery efficiency and various atomization satisfactions. The substrate (1) is deposited with the low oxygen content film and the passive film (3), so that the non-porous areas are the heating surface. Combining with the controllable fluidic transferring channels (4), uniform atomization is realized for different kinds of e-liquids and other liquids, without coking, ceramic particle emission or any heavy metals.

### Embodiment 2

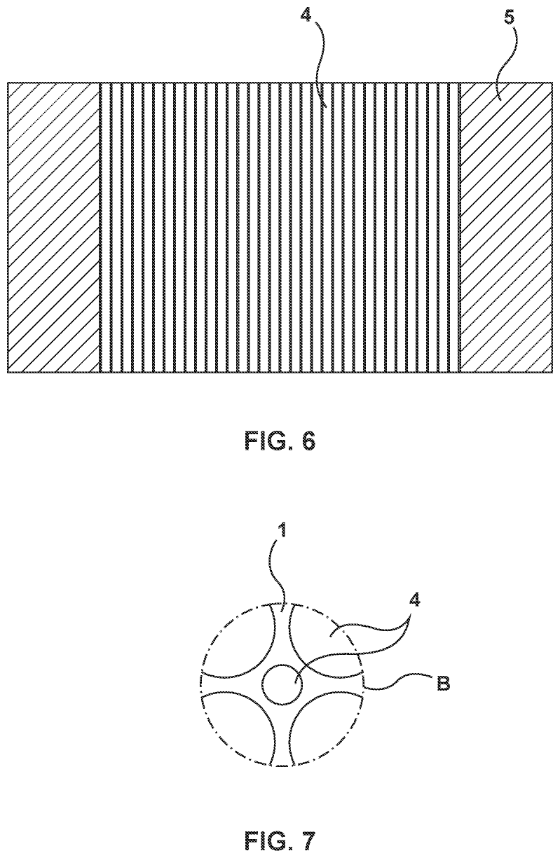

Referring to FIGS. 1, 5, 6 and 7, the disclosure further provides an atomization core comprising a substrate (1) made of monocrystalline alumina, on which is deposited a film with low oxygen content, which is a titanium or tantalum oxide film. The thickness of the film with low oxygen content is 4 μm, on which is deposited a passive film (3), namely an Au film. The thickness of the passive film (3) is 15 nm. The substrate (1) is formed with fluidic transferring channels (4) therein. The diameter of each of the perforations of the fluidic transferring channels (4) is 100 μm, which is processed by laser or drilled mechanically. The array of the perforations of the fluidic channels (4) is arranged in a rectangular shape. The spacing between the walls of the adjacent perforations is 200 μm. In some examples, as shown in FIG. 7, a perforation with a smaller diameter may be located between a group of four larger perforations.

Electrodes (5) are formed on both ends of the substrate (1), are made of safe conductive paste, and connected to a battery. The back of the substrate (1) is formed with a groove (6), which is connected to the fluidic transferring channels (4). The film with low oxygen content and the passive film (3) deposited between the walls of the adjacent perforations form a uniform temperature field and uniform vapor nucleation centers. Combining controllable liquid fluidic and air fluidic channels during the atomization process, the aerosol generation is also controlled to achieve better nicotine delivery efficiency and various atomization satisfactions. The substrate (1) is deposited with the film with low oxygen content and the passive film (3), so that the non-porous areas are the heating surface. Combining with the controllable fluidic transferring channels (4), uniform atomization is realized for different kinds of e-liquids and other liquids, without coking, ceramic particle emissions or any heavy metals.

How it works: when the electrodes (5) are energized, the film with low oxygen content of the heating layer (2) and the passive film (3) deposited on the substrate (1) between the perforation walls form a uniform temperature field and uniform vapor nucleation centers. As the substrate (1) is made of monocrystalline alumina, with the film with low oxygen content and the passive film (3) deposited on the surface, and the diameter of the perforations of the fluidic transferring channels (4) is uniform, uniform atomization is realized, without coking or ceramic particle emission. For some e-liquids transferring through the fluidic transferring channels (4) and the walls thereof, the atomization nucleation processing and the dynamic growth process after nucleation are more accurately controlled, so that the particle size and composition, quantity and temperature of atomized aerosol can be controlled or tailored according to specific atomization requirements, and the nicotine delivery efficiency can be improved to a certain extent.

Although embodiments of the disclosure have been shown and described, a person skilled in the art can easily understand that various changes, modifications, replacements and variations can be made to these embodiments within the principles of the present disclosure, and the scope of the disclosure is defined by the appended claims and their equivalents.

What is claimed is:

1. An atomization core comprising:
a substrate having a back side and a heating side;
a plurality of fluidic transferring channels comprising perforations defined in the substrate, the perforations extending from the back side to the heating side of the substrate; and
a heating layer deposited on the heating side of the substrate between the perforations.

2. The atomization core according to claim 1, wherein the perforations have a uniform diameter.

3. The atomization core according to claim 1, wherein the perforations have a uniform spacing.

4. The atomization core according to claim 1, further comprising a passive layer deposited on the heating layer.

5. The atomization core according to claim 1, further comprising electrodes formed on the substrate.

6. The atomization core according to claim 1, wherein each perforation has a diameter of less than 250 μm.

7. The atomization core according to claim 1, wherein each perforation is spaced from an adjacent perforation by less than 500 μm.

8. The atomization core according to claim 1, wherein each perforation is defined by a wall and wherein the heating layer is deposited on the heating side of the substrate between the walls of the perforations.

9. The atomization core according to claim 1, wherein the substrate is made from monocrystalline alumina.

10. The atomization core according to claim 1, wherein the substrate is made from monocrystalline silicon.

11. The atomization core according to claim 1, wherein the substrate is made from polycrystalline silicon.

12. The atomization core according to claim 1, wherein the substrate is made from a dense ceramic material.

13. The atomization core according to claim 1, wherein the heating layer comprises a pure metal film.

14. The atomization core according to claim 1, wherein the heating layer comprises an alloy film.

15. The atomization core according to claim 1, wherein the heating layer comprises a film with low oxygen content.

16. The atomization core according to claim 1, wherein the heating layer comprises a titanium oxide film.

17. The atomization core according to claim 1, wherein the heating layer comprises a tantalum oxide film.

18. The atomization core according to claim 4, wherein the passive layer comprises one of an inert metal, alloy, or compound film.

19. An atomization core comprising:
a substrate having a back side and a heating side;
a plurality of fluidic transferring channels comprising perforations having a uniform diameter and a uniform spacing defined in the substrate, the perforations extending from the back side to the heating side of the substrate; and
a heating layer deposited on the heating side of the substrate between the perforations.

20. An atomization device comprising an atomization core, wherein the atomization core comprises:
a substrate having a back side and a heating side;
a plurality of fluidic transferring channels comprising perforations defined in the substrate, the perforations extending from the back side to the heating side of the substrate; and
a heating layer deposited on the heating side of the substrate between the perforations.
